(19) United States
(12) Patent Application Publication
(10) Pub. No.: US 2024/0242794 A1
(43) Pub. Date: Jul. 18, 2024

(54) METHOD FOR OPERATING A LABORATORY AUTOMATION SYSTEM, LABORATORY AUTOMATION SYSTEM, AND LABORATORY IN-VITRO DIAGNOSTIC SYSTEM

(71) Applicant: Roche Diagnostics Operations, Inc., Indianapolis, IN (US)

(72) Inventor: Josep Fernandez, Sant Cugat del Valles (ES)

(73) Assignee: Roche Diagnostics Operations, Inc., Indianapolis, IN (US)

(21) Appl. No.: 18/560,126

(22) PCT Filed: Apr. 28, 2022
(86) PCT No.: PCT/EP2022/061347

§ 371 Date: Nov. 10, 2023

(30) Foreign Application Priority Data
May 11, 2021 (EP) 21382431.1

(51) Int. Cl. G16H 10/40; G01N 35/00; G01N 35/04

(52) U.S. Cl. CPC G16H 10/40; G01N 35/00871; G01N 35/04; G01N 2035/0091

(57) ABSTRACT

A method for operating a laboratory automation system, comprising, a transport system, a plurality of transfer devices; a portable device; and a plurality of data communication modules. The method comprises: receiving device information for the devices in the portable device, the device information being indicative a device identification; receiving a first user input in the portable device, the user input being indicative of a selection of a first device from the plurality of devices; in response to receiving the first user input, pairing the first device with the transport system for data communication in operation of the first device for at least one of sample pre-analytics and sample analysis; and providing first pairing information in the portable device, the first paring information being indicative of the first device and the distribution system being paired successfully. Further, a laboratory automation system and a laboratory in-vitro diagnostics system are provided.

The present disclosure refers to a method for operating a laboratory automation system. Further, the present disclosure refers to a laboratory automation system and a laboratory in-vitro diagnostic system.

### BACKGROUND

Laboratory automation systems are applied, in particular, for determining samples, for example samples of a bodily fluid, essentially automatically. The samples are typically received in sample vessels or containers, which are processed via a laboratory automation system.

Such laboratory automation systems may comprise several units. Laboratory automation systems usually comprise several laboratory stations such as, for example, a pre-analytical, an analytical and/or a post-analytical station. Typically, the containers are transported between different stations of the system via a sample distribution system. The sample vessels may be moved along a line of processing for processing, wherein the sample vessels and/or sample vessel carriers are moved by means of a transport device having one or more actuators and actuator drivers or driving devices for driving the actuator(s). For example, the sample vessels may be moved or relocated from a first working station to a second working station provided in the line of processing in the system. The working stations may also be referred to as working locations.

Document WO 2011/138 448 A1, for example, relates to a system for transporting containers between different stations, wherein the containers are accommodated in container carriers. The system comprises a control unit, which controls the transportation of the container carriers, a transporting surface, which is subdivided into sub-surfaces and on which the container carriers may be arranged in a movable manner, and drive means, wherein the drive means are activated by the control unit and one drive means in each case is assigned to one sub-surface in each case, wherein a drive means in each case is designed in order to provide an associated container carrier with driving power.

Laboratory automation systems may also comprises at least one transfer device to transfer a sample item, wherein the sample item may be a container carrier, a sample container, part of the sample and/or the complete sample, between the transport plane and a laboratory station.

When installing a new laboratory system, secure connections between a transport management system and several units of the system are needed. After a new laboratory system is assembled, the transport management system may be provided with trusted connections with a plurality of units of the system. However, the transport management system may be unaware of the interrelationships between the units. Thus, for example, the devices of the system should be linked, e.g. by a user, to the transfer system and/or transport system interfaces. The identification of each unit is needed in order to establish adequate links between the several units. Typically, this is done manually or by taking pictures of each unit (identification), which is neither efficient nor user friendly and is also error-prone, which might cause serious configuration problems due to incorrect identifications and delays in installations of laboratories.

Document US 2016/0 321 421 A1 discloses a point of care (POC) testing system and a method for configuration of a POC testing system that provide a workflow solution for the configuration management of POC analyzers. The POC system and method provide for convenient assisted workflows that enable efficient replacement of POC analyzers and enable traceable relocation of POC analyzers within, for example, a hospital.

In document WO 2019/115 465 A1, a system of medical devices is disclosed. The system comprises: a medical device data management system; a plurality of medical devices communicatively connected to the medical device data management system; each medical device being operable to analyze one or more samples of biological material; and to communicate information about an operational state of the medical device to the medical device data management system; a plurality of portable electronic devices, each operable to be carried by an operator, each portable electronic device communicatively connectable to the medical device data management system and configured to receive, from the medical device data management system, information indicative of an operational state of respective medical devices and to display the received information in respect of one or more selected ones of the medical devices.

Document US 2012/233 679 A1 refers to a system and method for initiating and maintaining a secure wireless communication between a wireless analyzer and a target network (e.g., a hospital network connected to a LIS and/or HIS). The disclosure of this document provides processes and systems for securely networking a wireless analyzer with a Wi-Fi network without the need for an operator or user to engage in manual initiation steps on, or through, the wireless analyzer.

Document US 2021/0 123 935 A1 discloses an automated analysis system. In the automated analysis system, a tablet terminal has a terminal information management unit and a terminal display unit. The terminal information management unit acquires device information showing the state status of a device from automatic analyzers, and generates a status confirmation screen showing the device status of the automatic analyzer. The terminal display unit displays the status confirmation screen. The status confirmation screen is provided with a status information screen showing the device status of one automatic analyzer and a device switch button configured to switch the status information screen to a status information screen corresponding to another other automatic analyzer. When the switch button is selected, the terminal information management unit generates a status confirmation screen for an automatic analyzer corresponding to the selected switch button, and displays the status confirmation screen on the display unit.

WO 2014/189 706 A2 refers to a method and a system for configuring computing devices using mobile workspace context based on proximity to locations. A mobile computing device determines that the device is proximate to a location, another device, or an individual associated with an enterprise system. The mobile computing device then receives a mobile workspace context associated with the location, device, or individual, such as one or more specific wireless networks, enterprise applications, and/or documents, and may configure the device based on the received mobile workspace context. Additional methods and systems are described for transmitting and receiving sets of device capabilities between multiple devices, establishing communication sessions, and sharing various capabilities between devices.

In US 2013/232 425 A1 a computer-implemented method for providing access control to a function of an analyzer is disclosed. A user's log-in data is received from a first data entry terminal. Information descriptive of a position of the first data entry terminal is obtained. It is determined whether the position of the first data entry terminal is within a physical operating (PO) area of the analyzer. A user activatable position sensitive (PS) GUI element is displayed on the first data entry terminal if the position of the first data entry terminal is determined to be within the PO area. The activatable PS GUI element triggers, upon activation, the execution of the function. The execution of the function comprises the execution of a physical action by the analyzer. The display of the user activatable PS GUI element is otherwise prohibited or a non-activatable PS GUI element indicative of the function is displayed.

### SUMMARY

It is an object of the present disclosure to provide improved technology for operating a laboratory automation system. In particular, it is an object of the present disclosure to provide improved technology for efficiently and flexibly installing a laboratory automation system.

For solving the problem, a method for operating a laboratory automation system according to the independent claim 1 is provided. Further, a laboratory automation system according to the independent claim 14 is provided. In addition, a laboratory in-vitro diagnostic system is provided according to the independent claim 15. Further embodiments are disclosed in dependent claims.

According to one aspect, a method for operating a laboratory automation system is provided. The laboratory automation system comprises: a plurality of devices provided in a laboratory area configured for at least one of sample pre-analytics and sample analysis; a transport system having a plurality of carriers configured to carry one or more sample containers and configured to transport the plurality of carriers to the plurality of devices; a plurality of transfer devices each assigned to at least one of the plurality of devices and configured to transfer at least one of a carrier from the plurality of carriers and a sample container received in the at least one carrier between the transport system and the at least one device; a portable device having one or more data processors and a user interface comprising a display configured to output image data, and an input device configured to receive user input; and a plurality of data communication modules configured for wireless data communication, wherein each of the plurality of data communication modules is assigned to at least one of a device from the plurality devices, the transport system, a transfer device from the plurality of transfer devices, and the portable device. The method comprises: receiving device information for the plurality of devices in the portable device, the device information being indicative of a device identification, receiving a first user input in the portable device, the user input being indicative of a selection of a first device from the plurality of devices, in response to receiving the first user input, pairing the first device with the transport system for data communication in operation of the first device for at least one of sample pre-analytics and sample analysis, and providing first pairing information in the portable device, the first paring information being indicative of the first device and the distribution system being paired successfully.

According to another aspect, a laboratory automation system is provided, comprising: a plurality of devices provided in a laboratory area configured for at least one of sample pre-analytics and sample analysis, a transport system having a plurality of carriers configured to carry one or more sample containers and configured to transport the plurality of carriers to the plurality of devices; a plurality of transfer devices each assigned to at least one of the plurality of devices and configured to transfer at least one of a carrier from the plurality of carriers and a sample container received in the at least one carrier between the transport system and the at least one device; a portable device having one or more data processors and a user interface comprising a display configured to output image data, and an input device configured to receive user input; and a plurality of data communication modules configured for wireless data communication, wherein each of the plurality of data communication modules is assigned to at least one of a device from the plurality devices, the transport system, a transfer device from the plurality of transfer devices, and the portable device. The laboratory automation system is configured to: receive device information for the plurality of devices in the portable device, the de-vice information being indicative of a device identification; receive a first user input in the portable device, the user input being indicative of a selection of a first device from the plurality of devices; in response to receiving the first user input, pair the first device with the transport system for data communication in operation of the first device for at least one of sample pre-analytics and sample analysis; and provide first pairing information in the portable device, the first paring information being indicative of the first device and the distribution system being paired successfully.

According to still another aspect, a laboratory in-vitro diagnostic system is provided, which comprises this laboratory automation system.

The transport system may be provided with a plurality of transport modules each having a module transport surface. A driving device may be configured to move the (sample vessel) carrier(s) on the (module) transport surface. The transport plane may be formed by module transport surfaces of transport modules of an arrangement of transport modules. The control device may be configured to control operation of the driving device. To each of the transport modules a module-ID may be assigned. The transport system may be managed by a transport management system.

The display of the portable device may be provided with an output device. The output device may further comprise a screen, in particular a touch screen. The screen may be the display. Alternatively or in addition, the output device may comprise a speaker. The input device may comprise a touch screen, a keyboard, a microphone (with speech recognition), a joystick, a button, and/or an adjusting wheel. The portable device may be a portable computer. In particular, the portable device may be a tablet or a smartphone. First pairing information may be outputted for user information. First pairing information may be outputted via the output device.

Device pairing is a form of information registration for linking or connecting devices or modules for data communication. Thus, the device pairing may also be referred to as “assignment for data communication”. By registering device information (pairing) between devices, the devices or modules may connect for data communication using at least one of wireless and wired data communication.

The transfer device assigned to or associated with at least one of the plurality of devices, by such functional or operational assignment, provides for transferring the at least one of the carrier from the plurality of carriers and the sample container received in the at least one carrier between the transport system and the at least one device.

The portable device may be carried by a user or a robot. The portable device may be remotely operated.

The pairing may comprise pairing the pairing comprises pairing a first transfer device assigned to or provided with the first device with the transport system for data communication in operation of the first device for at least one of sample pre-analytics and sample analysis. By such pairing the first transfer device may be assigned a first location of the transport system (“assignment of location”). The first location of the transport system may be located opposite the first transfer device/the first device. The first location of the transport system may be a location of a first transport module within the transport system. The first transport module may have or be assigned a first module-ID. The first pairing information may be indicative of a first transfer device identification (transfer-device-ID) and the first transport module identification (module-ID). The first pairing information may be indicative of a first device identification (device-ID) and the first transport module identification (module-ID).

The first device from the plurality of devices may comprise the first transfer device. The first transfer device may be an inherent component of the first device. In this case, the first transfer device identification (transfer device-ID) and the first device identification (device-ID) may be equal or derivable from each other. The first device and the first transfer device may be inherently assigned to each other.

The pairing may comprise pairing the first device with a first transport system interface of the transport system, thereby assigning the first transport system interface to the first device.

The pairing may comprise pairing the first transfer device with the first transport system interface for data communication in operation of the first device for at least one of sample pre-analytics and sample analysis.

The pairing may further comprise pairing the first transport system interface with the transport system, thereby, for example, assigning the first transport system interface to the first location of the transport system.

The method may further comprise assigning a first interface identification to the first transport system interface, wherein the first pairing information is indicative of a first device identification assigned to the first device and the first interface identification. By such assignment, the first device identification is provided for identifying the first device in the laboratory automation system. Also, by assignment of the first interface identification to the first transport system interface, the first transport system interface can be identified in the laboratory automation system. With respect to such embodiment or alternative embodiments, identification data indicative of one or more identifications may be stored in a memory of the laboratory automation system.

The method may further comprise assigning the first interface identification to the first transport system interface and a first transfer device identification to the first transfer device, wherein the first pairing information is indicative of the first interface identification and the first transfer device identification.

In general, the laboratory automation system comprises a plurality of system units or system elements. A unit or element may be, for example, at least one of a device, a transfer device, a transport system interface, and (a section/module of) the transport system. Each unit may have assigned an individual ID, thereby, providing a unique identifier for the unit or element at least in the laboratory automation system. Pairing may comprise pairing a first unit with a second unit different from the first unit to establish a communication link between the first unit and the second unit.

Pairing may comprise pairing the first unit with the second unit different from the first unit to assign the first unit to the second unit for data communication. The first unit and the second unit may correspond to a first operating unit. The first operating unit may comprise the first device, the first transfer device, the first transport system interface, the first transport module, and/or a first section of the transport system. The first section of the transport system may be the transport system or a part of the transport system, located opposite the first device, the first transfer device, and/or the first transport system interface.

The receiving of the device information may comprise receiving device information being indicative of a device location in the laboratory area. For example, first device information may indicative of a first location of the first device within the laboratory system. For example, the laboratory automation system may be assigned a coordinate system. Device location may be determined by coordinates of the coordinate system.

The device or some other unit/element of the laboratory automation system may determine its position on its own and may broadcast location information indicative of the position. Alternatively, the device or some other unit/element of the laboratory automation system may transmit a pre-defined signal. Via the pre-defined signal and a trilateration method the position of the device may be determined externally and may be provided for the portable device. The calculation may be performed by a transport management system. For the trilateration method, three receivers of the pre-defined signal may be provided. Through the receivers distances between the at least one unit and the receivers may be determined. However, also more than three receivers may be provided.

The receiving of the first user input may comprise: determining a local position of the portable device within the laboratory area; providing a device selection menu indicative of one or more devices from the plurality of devices, the one or more devices being located in the vicinity of the local position of the portable device; outputting the device selection menu via the display; and, in the portable device, receiving a first user input indicative of a selection of the first device from the one or more devices provided in the device selection menu.

The local position of the portable device may be determined by trilateration (e.g., via GPS) and/or triangulation. Alternatively or in addition, the receiving of the first user input may comprise: determining broadcasted (pre-defined) signals from the plurality of devices; providing a device selection menu indicative of one or more devices from the plurality of devices, the one or more devices having a determined signal strength that is greater than a threshold value A; outputting the device selection menu via the display; and, in the portable device, receiving a first user input indicative of a selection of the first device from the one or more devices provided in the device selection menu.

The device selection menu may only comprise a single device form the plurality of devices. The single device may be the device nearest to the portable device. The single device may correspond to the strongest determined signal from the plurality of signals broadcasted from the plurality of devices. The single device may be the device from the plurality of devices for which the distance to the location of the portable device is the smallest, i.e. the absolute value of the difference between the device position and the portable device position is the smallest.

The one or more devices located in the vicinity of the local position of the portable device may be devices that are located in a limited area around the local position of the portable device, e.g., in a circle/sphere defined by a radius in the center of which the portable device is located.

The device selection menu may provide the devices (e.g., in form of device-IDs) in an ordered manner, e.g. in a table. The order may correspond to one of the distance of the devices to the portable device (ascending or descending), and signal strength of the broadcasted signals received in the portable device. Already linked/paired devices may be marked or prevented from displaying in the device selection menu.

The local position of the portable device within the laboratory area may be determined more than once. The local position of the portable device within the laboratory area may be determined regularly, e.g. at regular (time) intervals. In this way, the local position of the portable device may be tracked while the portable device is moving within the laboratory area.

The receiving of the first user input may comprise: providing an interface selection menu indicative of one or more transport system interfaces available for pairing with the one or more devices from the device selection menu; outputting the interface selection menu via the display; and, in the portable device, receiving a first interface user input indicative of a selection of the first transport system interface from the one or more transport system interfaces provided in the interface selection menu.

Alternatively or in addition, the receiving of the first user input may comprise: determining a local position of the portable device within the laboratory area; providing an interface selection menu indicative of one or more transport system interfaces from the plurality of transport system interfaces, the one or more transport system interfaces being located in the vicinity of the local position of the portable device; outputting the interface selection menu via the display; and, in the portable device, receiving a first user input indicative of a selection of the first transport system interface from the one or more transport system interfaces provided in the interface selection menu.

In another example, the receiving of the first user input may comprise: determining broadcasted signals from the plurality of transport system interfaces: providing an interface selection menu indicative of one or more transport system interfaces from the plurality of transport system interfaces, the one or more transport system interfaces having a determined signal strength that is greater than a threshold value B; outputting the interface selection menu via the display; and, in the portable device, receiving a first user input indicative of a selection of the first transport system interface from the one or more transport system interfaces provided in the interface selection menu.

The interface selection menu, in an example, may only comprise or show a single transport system interface which may be the transport system interface determined to be nearest to the one or more devices. The single transport system interface may correspond to the strongest determined signal from the plurality of signals broadcasted from the plurality of transport system interfaces. The single transport system interface may be the transport system interface from the plurality of transport system interfaces for which the distance to the location of the portable device is the smallest, i.e. the absolute value of the difference between the transport system interface position and the portable device position is the smallest. The single transport system interface may be the transport system interface that is assigned to the device selected via the device selection menu.

The one or more transport system interfaces located in the vicinity of the local position of the portable device may be transport system interfaces that are located in the limited area around the local position of the portable device.

The interface selection menu may provide the one or more transport system interfaces in an ordered manner, e.g. in a table. The order may correspond to the distance of the one or more transport system interfaces to the portable device (ascending or descending). Already linked/paired transport system interfaces may be marked or not displayed in the interface selection menu.

The receiving of the first user input may comprise: providing a transfer device selection menu indicative of one or more transfer devices available for pairing with the one or more devices from the device selection menu and/or the one or more transport system interfaces from the interface selection menu; outputting the transfer device selection menu via the display; and, in the portable device, receiving a first transfer device user input indicative of a selection of the first transfer devices from the one or more transfer devices provided in the transfer device selection menu.

Alternatively or in addition, the receiving of the first user input may comprise: determining a local position of the portable device within the laboratory area; providing a transfer device selection menu indicative of one or more transfer devices from the plurality of transfer devices, the one or more transfer devices being located in the vicinity of the local position of the portable device; outputting the transfer device selection menu via the display; and, in the portable device, receiving a first user input indicative of a selection of the first transfer device from the one or more transfer devices provided in the transfer device selection menu.

Alternatively or in addition, the receiving of the first user input may comprise: determining broadcasted signals from the plurality of transfer devices; providing a transfer device selection menu indicative of one or more transfer devices from the plurality of transfer devices, the one or more transfer devices having a determined signal strength that is greater than a threshold value C; outputting the transfer device selection menu via the display; and, in the portable device, receiving a first user input indicative of a selection of the first transfer device from the one or more transfer devices provided in the transfer device selection menu.

The transfer device selection menu may comprise only a single transfer device. The single transfer device may be the nearest transfer device. The single transfer device may correspond to the strongest determined signal from the plurality of signals broadcasted from the plurality of transfer devices. The single transfer device may be the transfer device from the plurality of transfer devices for which the distance to the location of the portable device is the smallest, i.e. the absolute value of the difference between the transfer device position and the portable device position is the smallest. The single transfer device may be the transfer device that is assigned to the device selected via the device selection menu and/or to the transport system interface selected via the interface selection menu.

The one or more transfer devices located in the vicinity of the local position of the portable device may be transfer devices that are located in the limited area around the local position of the portable device.

The transfer device selection menu may provide the one or more transfer devices in an ordered manner, e.g. in a table. The order may correspond to the distance of the one or more transfer devices to the portable device (ascending or descending). Already linked/paired transfer devices may be marked or not displayed in the transfer device selection menu.

A global selection menu may comprise at least one of the device selection menu, the interface selection menu, and the transfer device selection menu. At least one of the selection menus may be provided such that the user may select at least two units to be paired.

The paring information may be indicative of the thus selected first device, first transfer device, and/or first transport system interface.

The receiving of the device information may comprise receiving device signals indicative of device information and broadcasted by the plurality of devices while the portable device is moving through the laboratory area. The receiving of the device information may comprise receiving transfer device signals and/or transport system interface signals.

The broadcasted signals by the system units or elements, such as the devices, may be range limited. By moving the portable device through the laboratory area the system unit information, such as device information, may be received step by step (depending on whether or not corresponding individual signals may be received).

The method may further comprise outputting a laboratory layout via the display, the laboratory layout being indicative of the arrangement of the plurality of devices and the transport system in the laboratory area. The laboratory layout may correspond to a map of the laboratory. The laboratory layout may be indicative of an arrangement of the plurality of units in the laboratory area.

At least one of the selection menus may be coupled with the laboratory layout. At least one of the selection menus and the laboratory layout may be shown on the display simultaneously. At least one unit-ID may be (visually) presented in the laboratory layout, e.g. at the positions of the corresponding unit. The user may select at least one unit-ID (visually) presented on the display (e.g., by tapping on the unit-ID). By selecting (e.g., by tapping on) two different units, the user may select to pair these two units.

The outputting may comprise outputting the laboratory layout with second marking information being indicative of one or more non-paired units (e.g., devices) from the plurality of devices. The second marking information may be visually overlaid on the display over the laboratory layout. The second marking information may comprise one or more second markers. Each second marker of the one or more second markers may correspond to one non-paired unit, e.g., one of the non-paired units of the corresponding menu, such as one non-paired device. A certain second marker may correspond to a certain non-paired system unit or element (e.g., device). The certain second marker may be located at a position of the corresponding certain non-paired system unit or element (e.g., device) in the output laboratory layout. The second marker may be an arrow. The second marker may be a two dimensional form. The form of the second marker may correspond to the form of the corresponding non-paired unit (e.g., device) in the output laboratory layout. In particular, the form of the second marker may be equal to the form of the corresponding non-paired unit (e.g., device) in the output laboratory layout. The second marker may be colored. The second marker may be semitransparent. The second marker may comprise a symbol and/or a pictogram, in particular a tick. The second marker may comprise the unit-ID of the corresponding non-paired system unit or element (e.g., device). The color of the second marker may be indicative of a unit type (device, transfer device, transport system interface, transport module, transport system). In particular, second markers of selected system units or elements (e.g., of the non-paired units of at least one of the menus) may comprise a symbol and/or a pictogram, in particular a tick.

The outputting may comprise outputting the laboratory layout with first marking information being indicative of one or more paired devices from the plurality of devices being successfully paired with the transport system.

The IDs of successfully paired system units or elements may be prevented from outputting (e.g., may not be shown on the display). The device-IDs of the one or more paired devices may not be outputted (e.g., may not be shown on the display). The first marking information may be visually overlaid on the display over the laboratory layout. The first marking information may comprise one or more first markers. Each first marker of the one or more first markers may correspond to one paired unit, in particular one paired device. A certain first marker may correspond to a certain paired unit (e.g., device). The certain first marker may be located at a position of the corresponding certain paired unit (e.g., device) in the output laboratory layout. The first marker may be an arrow. The first marker may be a two dimensional form. The form of the first marker may correspond to the form of the corresponding paired unit (e.g., device) in the output laboratory layout. In particular, the form of the first marker may be equal to the form of the corresponding paired unit (e.g., device) in the output laboratory layout. The first marker may be colored. The first marker may be semitransparent. The first marker may comprise a symbol and/or a pictogram, in particular a tick. Adjacent first markers may be combined into a combined first marker. All or at least two first markers may have the same color.

The markers can help the user to know what has already been done (paired) and what still needs be done (similar to a progress bar).

The method may further comprise (i) providing protocol information in the portable device, the protocol information being indicative of the one or more paired devices, and (ii) transmitting the protocol information to a central backend device in response to user confirmation input received in the portable device.

The central backend device may be provided with or correspond to the transport management system. The transmitting may only be permitted if all devices are paired. Alternatively the transmitting may be permitted anytime, in particular when a new device is paired. The transmitting may be executed automatically or in response to a user input. The protocol information may be indicative of device identification(s) (device-ID(s)), interface identification(s) (interface-ID(s)), transfer device identification(s) (transfer device-ID(s)), and/or transport module identification(s) (module-ID(s)). The protocol information may be indicative which units are paired/linked with each other and/or which units are assigned to each other. The protocol information may be indicative of groups comprising device identification(s) (device-ID(s)), interface identification(s) (interface-ID(s)), transfer device identification(s) (transfer device-ID(s)), and/or transport module identification(s) (module-ID(s)). The IDs of one group may correspond to mutually paired/linked/assigned units. Each group may corresponded to one device. The protocol information may be indicative of a percentage of units already paired compared to the total number of units to be paired (progress in %).

The method may be executed via a software on the portable device. The software solution may be in the form of an APP, a web interface, and/or any other type of software solution. The portable device may comprise a navigation tool. The user may be navigate through the laboratory by means of the portable device. The navigation may be based on the navigation tool and the laboratory layout. Pairing the first device with the transport system may comprise providing information, e.g. to the transport device or the transport management system, indicative of where the carrier(s) are to be delivered (at a certain processing step). Via the portable device, changes to the laboratory layout may be performed.

The embodiments described above with respect to the method for operating a laboratory automation system may be provided for the laboratory automation system and/or the laboratory in-vitro diagnostic system, accordingly.

### DESCRIPTION OF FURTHER EMBODIMENTS

In the following, embodiments, by way of example, are described with reference to figures. In the figures show:

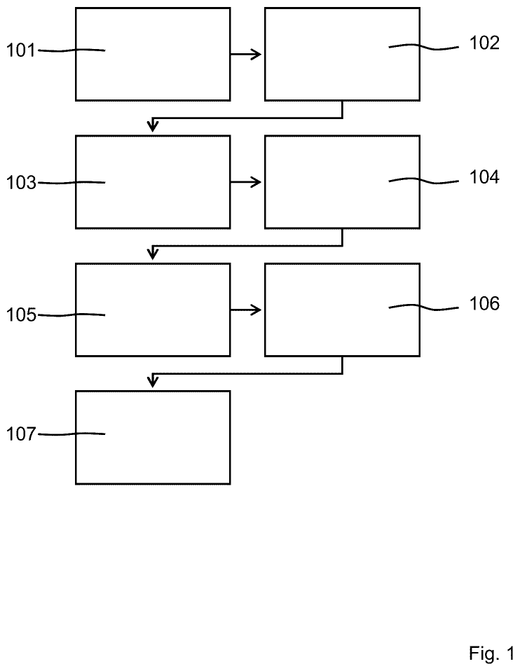

FIG. 1 a flowchart of one example of the method for operating the laboratory system;

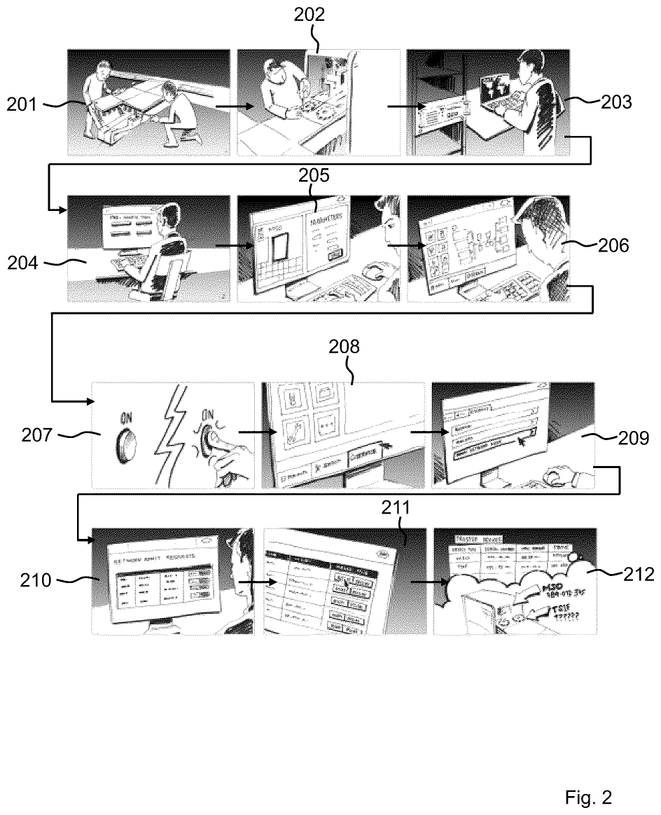

FIG. 2 a flowchart for an exemplary procedure for preinstalling the laboratory system;

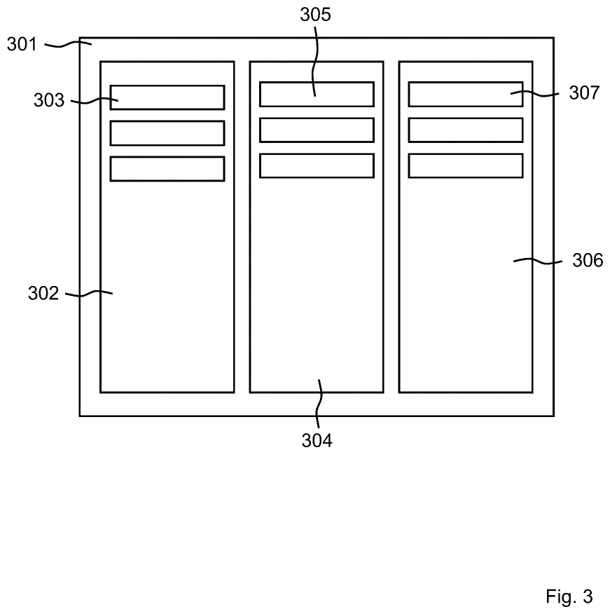

FIG. 3 a graphical representation of an exemplary menu for selecting several units;

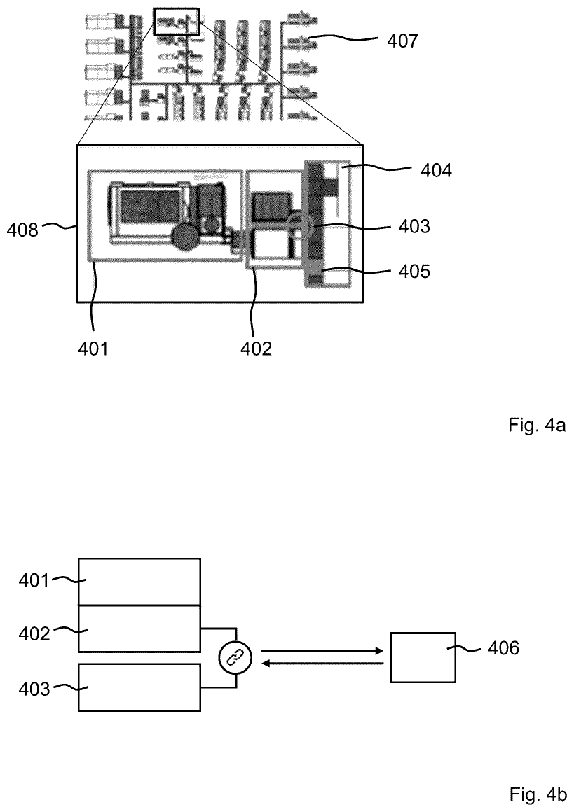

FIG. 4a a graphical representation of the laboratory system;

FIG. 4b a graphical representation of pairing the first transport system interface with the first transfer device;

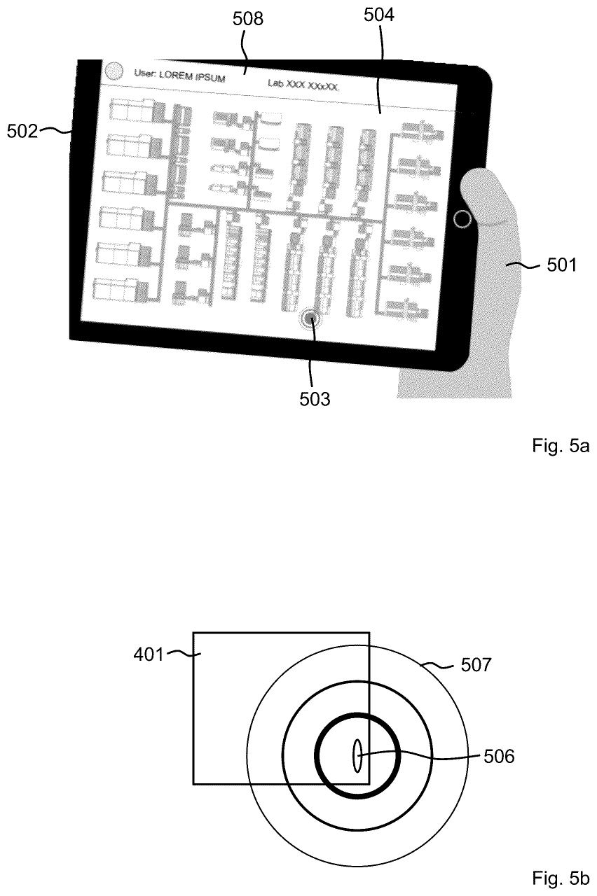

FIG. 5a a graphical representation of the laboratory layout shown on the display of the portable device;

FIG. 5b a graphical representation of one device broadcasting a corresponding signal;

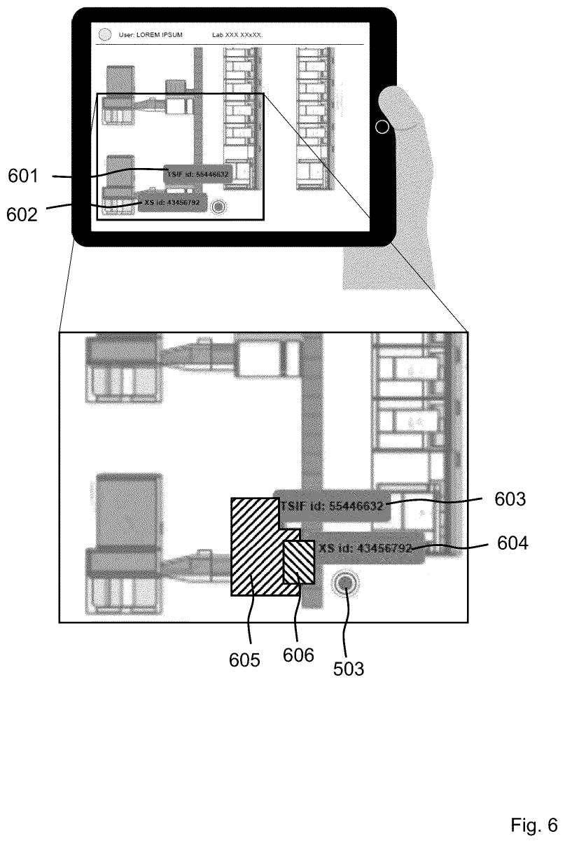

FIG. 6 a graphical representation of the laboratory layout shown on the display of the portable device with marking information;

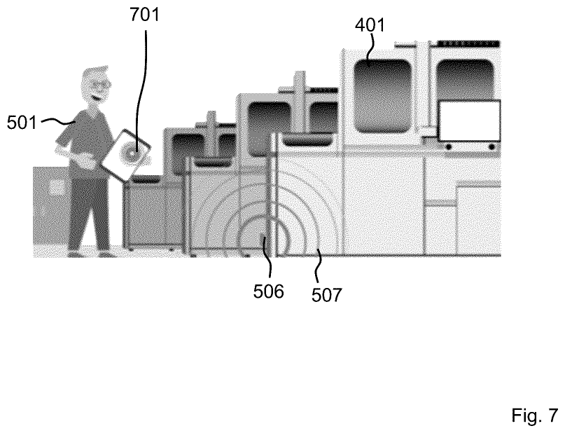

FIG. 7 a graphical representation of receiving device information step by step by moving the portable device through the laboratory area;

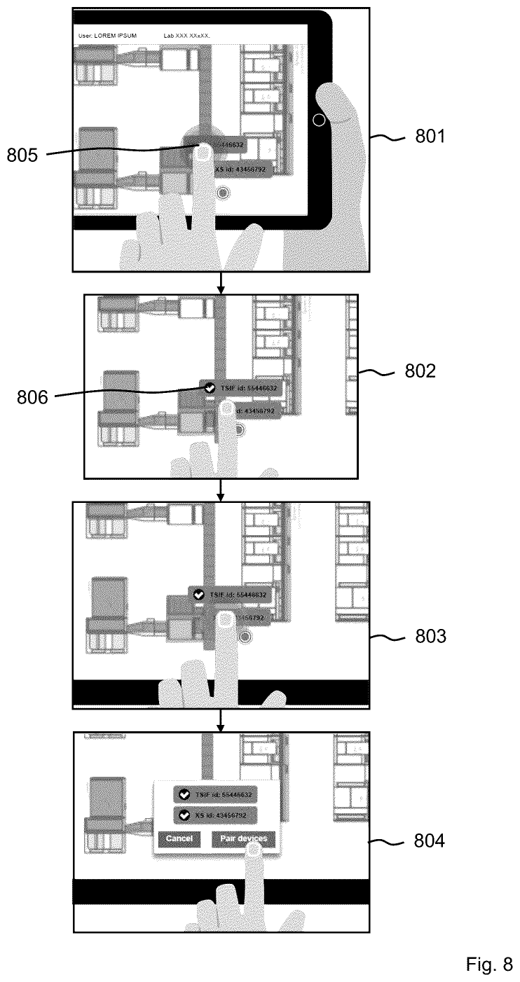

FIG. 8 a flowchart for an exemplary procedure for selecting two units for pairing;

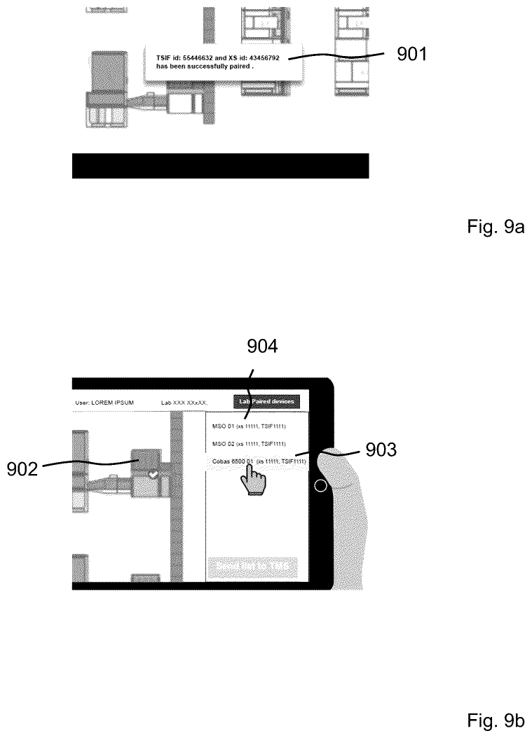

FIG. 9a a first graphical representation of marking information;

FIG. 9b a second graphical representation of marking information; and

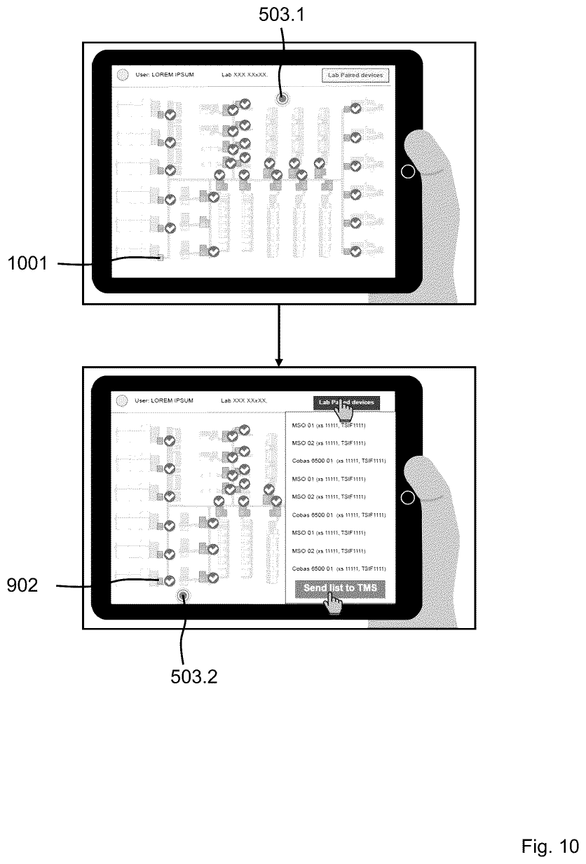

FIG. 10 an exemplary procedure for pairing a remaining unit;

Referring to FIGS. 1 to 5, a flowchart of one example of the method for operating a laboratory automation system 407 (see FIG. 4) is shown in FIG. 1.

According to the example, in step 101, a user 501 (see FIG. 5), e.g. a technician, may arrive at the laboratory automation system 407. The user 501 may carry the portable device 502. When arriving at the laboratory automation system 407, the user 501 may, in step 102, start an application on the portable device. In reaction, on the portable device 502, the map of the laboratory/the laboratory layout 504 may be shown on the display 508 of the portable device 502. In step 103, the application of the portable device 502 may locate the position of the portable device 502 in the laboratory automation system 407 and may present this position on the map of the laboratory 504 on the display 508, e.g. by means of a position marker 503. Each time the position of the device is determined, the map 504 and/or the location of the position marker 503 may be updated. In a fourth step 104, the user 501 may identify physical connected units in the laboratory automation system 407, in particular devices 401 that are connected to the transport system 404. These units may not be installed, i.e. may not be paired. The portable device may receive information for the plurality of units, in particular for the plurality of devices 401. In reaction, the user 501 may identify the first unit, in particular, the first device 401. The first unit may be a unit to be paired (with the system). The first unit may be a unit that is not installed yet. The selection may be made via a user input on the touch screen 508 in the application of the portable device 502.

In reaction, in step 105, a paring partner may be selected. Therefore, the transport system 404 may be selected (automatically). Another unit, e.g. the first transfer device 402, the first transport system interface 403, and/or the first module 405, may be selected. The paring partner may already be installed, may already be paired (with the system). In a sixth step 106, the selected paring partners may be paired. In step 107, the pairing information may be provided in the portable device 502 and optionally may be transmitted to the central backend device and/or the transport management system 406.

Before the method for operating the laboratory automation system 407 may be performed, it may be necessary to preinstall the laboratory automation system 407.

FIG. 2 shows a flowchart for an exemplary procedure for preinstalling the laboratory automation system 407. In preinstalling step 201, the transport modules 405 of the transport system 404 may be physically installed. In preinstalling step 202, the transfer devices 402 and the devices 401 may be physically installed. Afterwards, the laboratory automation system 407 may be physically installed, e.g. according to a LabLayout provided by a PreConfig server. In a third preinstalling step 203, the transport management system 406 server may be physically installed. The further preinstalling steps 204 to 206 may relate to the installation of the transport management system 406.

In preinstalling step 204, the transport management system 406 may be configured. In preinstalling step 205, the transport management system 406 may be provided with the laboratory layout 504, e.g. by downloading the LabLayout from the PreConfig server. In preinstalling step 206, the transport management system 406 may determine all (locations of the) transport system interfaces 403 of the transport system 404 of the loaded laboratory layout 504. As the transport system interfaces 403 may be inherent parts of the transport system 404, the transport system interfaces 403 may be inherently linked to the transport system 404.

In preinstalling step 207, the laboratory automation system 407 is supplied with power. In preinstalling step 208, the laboratory automation system 407 may be put into service mode.

In preinstalling step 209, a network node configuration may be selected by the user. In response, in preinstalling step 210, the user may be provided with a list of network nodes that are connected and try to establish a secure connection to the transport management system 406. The network nodes may correspond to units of the laboratory automation system 407. The user may then, in preinstalling step 211, select the network nodes she/he trusts, e.g. based on the serial number corresponding to the node. In preinstalling step 212, the trusted nodes, i.e. the trusted units, may be shown. However, the transport management system 406 might then know all of the units provided in the system 407, but the transport management system 406 may not know, after the preinstalling, the relationships between the units. In particular, the transport management system 406 may not know which units are to be assigned/paired to one another.

FIG. 3 shows a graphical representation of an exemplary menu for selecting several units. The shown menu may be the combined/global selection menu 301. The combined menu may comprise the device selection menu 302, 304 and a transfer device selection menu 306. The device selection menu 302, 304 may comprise a dive-ID selection menu 304 and a device name selection menu 302. At least one menu may be shown on the display 508 of the portable device 502. At least one menu may be provided to assign units to one another. Each menu may comprise/be a column of a table.

In the device name selection menu 302, e.g. the first column of the table, all names 303 of the trusted devices 401 may be provided. Alternatively, only devices 401 that are in the vicinity of the portable device 502 may be provided. For example, devices 401 that are no more than 4 m (2 m, 5 m, 6 m, 10 m) away from the portable device 502 may be provided. The devices 401 may be provided in an order (e.g., depending on their distance to the portable device 502). The closest device may be provided uppermost in the device name selection menu 302. Directly after the closest device, the second closest device may be provided in the menu device name selection menu 302 (and so on).

In the device-ID selection menu 304, e.g. the second column of the table, all IDs 305 of the trusted devices 401 may be provided. Alternatively, only device-IDs of devices 401 that are in the vicinity of the portable device 502 may be provided. Analogously to the device names, the device-IDs may be provided in an order. The device-IDs may be inherently assigned to the corresponding devices 401. In particular, each device 401 may broadcast a signal 507 indicative of its name and device-ID. The device name and device-ID may be comprised by the device information received by the portable device 502. Therefore, each provided device 401 in the device name selection menu 302 may automatically be linked to the corresponding device-ID. In the device selection menu 302, 304, corresponding device-IDs and device names may be provided next to each other, e.g. may be provided in the same line of the table. Alternatively, device-IDs and device names may not be inherently assigned to each other. In this case, the user may select, in the device selection menu 302, 304, for each device name a corresponding device ID. The device-ID selection menu 304 may comprise drop-down menus. To each device 401 in the device name selection menu 302, a drop-down menu in the device-ID selection menu 304 may be provided. The drop-down menu may provid device-IDs that may be assigned to the corresponding device name.

In the transfer device selection menu 306, e.g. the third column of the table, all trusted transfer devices 402 may be provided. Alternatively, only transfer devices 402 that are in the vicinity of the portable device 502 may be provided. For example, transfer devices 402 that are no more than 4 m (2 m, 5 m, 6 m, 10 m) away from the portable device 502 may be provided. Analogously to the device names, the transfer devices 402 may be provided in an order. The user may select, in the transfer device selection menu 306, a transfer device 402. Thereby, the user may link this selected transfer device 402 to the selected device 401 (device-ID and/or device name). The transfer device selection menu 306 may comprise drop-down menus. To each device 401 in the device name selection menu 302, a drop-down menu in the transfer device selection menu 306 may be provided. The drop-down menu may provided transfer devices (transfer device-IDs 307) that may be assigned to the corresponding device name.

The IDs of the units may be determined by taking a picture of the corresponding unit or by manually obtaining an ID that is printed on the corresponding unit. The picture may comprise the ID that is printed on the corresponding unit.

FIG. 4a shows a graphical representation of the laboratory automation system 407. The laboratory system 407 comprises the plurality of devices 401 that are configured for at least one of sample pre-analytics and sample analysis. Further, the laboratory automation system 407 comprises the transport system 404. The transport system 404 has a plurality of carriers configured to carry one or more sample containers and configured to transport the plurality of carriers to the plurality of devices 401. The transport system 404 may further comprise transport system interfaces 403, each of which may correspond to different devices 401. The transport system 404 may comprise or may be formed by a plurality of transport modules 405. For delivering the samples from the transport system 404 to the devices 401, transfer devices 402 may be provided. FIG. 4 further shows an example of the first operating unit 408. The first operating unit 408 in FIG. 4 comprises the first device 401, the first transfer device 402, the first transport system interface 403, the first section of the transport system 404, and the first transport module 405. The pairing may comprise paring any two different devices of the first operating unit 408.

FIG. 4b shows a graphical representation of pairing the first transport system interface 403 with the first transfer device 402. The paring may be performed by the user 501 via the portable device 502. The first device 401 and the first transfer device 402 may already be linked/assigned to each other (e.g., by prior pairing). Thus, pairing the first transport system interface 403 with the first transfer device 402 may be understood as pairing the first transport system interface 403 with the first device 401. Moreover, the first transport system interface 403 may inherently be linked/assigned to the transport system 404 (e.g., by prior pairing). Thus, pairing the first transport system interface 403 with the first transfer device 402 may be understood as pairing the transport system 404 with the first device 401. The pairing step shown in FIG. 4b may correspond to assigning the first transport system interface 403 to the first transfer device 402. The portable device 502 may transmit the information corresponding to this paring step to the transport management system 406. The transport management system 406 may store the interrelations between units thus paired. After this pairing, the transport system 404 and the transport management system 406, respectively, may be aware of where to deliver carriers.

FIG. 5a shows a graphical representation of the laboratory layout 504 shown on the display 508 of the portable device 502. For orientation of the user 501 in the laboratory automation system 407, the display 508 may show a map 504 of the laboratory automation system 407. The display 508 may show a map 504 of a section of the laboratory automation system 407. The portable device 502 may locate its position in the laboratory automation system 407. The position may be shown on the map of the laboratory 504 on the display 508, e.g. by means of the position marker 503. Each time the position of the device is determined, the map 504 and/or the location of the position marker 503 may be updated. Hence, the laboratory layout 504 may serve for navigation through the laboratory automation system 407.

FIG. 5b shows a graphical representation of one device 401 broadcasting a corresponding signal 507. The device may comprise a transmitter 506. The transmitter 506 may be a communication module 506 of the device. The signal 507 may broadcast information associated to the one device 401. The information may comprise information indicative of, the name of the device, the device-ID, the position of the device, and/or if the device is already paired. The single 507 may comprise a predefined signal with a predefined amplitude. The position of the one device 401 may be determined by receiving the predefined signal via at least three signal receivers. Depending on the received signal strength, distances between each of the receivers and the device 401 may be determined. In reaction, the position of the one device 401 may be determined. The above procedure analogously works vice versa, wherein the receivers are transmitters and the transmitter is a receiver. The position of further units and/or the portable device 502 may be determined analogously.

FIG. 6 shows a graphical representation of the laboratory layout 504 shown on the display 508 of the portable device 502 with marking information. In FIG. 6, the laboratory layout 504 also comprises the selection menu 301. The menu 301 comprises information indicative of the transfer device 402 and the transport system interface 403 that are located closest to the portable device 502. Specifically, the menu 301 in FIG. 6 comprises the transfer device-ID and the interface-ID corresponding to the transfer device 402 and the transport system interface 403 that are located closest to the portable device 502. The location of the portable device is shown by the position marker 503. FIG. 6 also shows marking information 601, 602 with markers. The markers 603 and 604 may comprise menu information indicative of units comprised by the menu 301. In the displayed laboratory layout 504, each marker may be located in the vicinity of the corresponding unit. In addition or alternative, the units in the vicinity of the portable device 502 may be highlighted with highlighting markers 605, 606. The closest transfer device 402 and transport system interface 403 may be highlighted with highlighting markers 605, 606. The first highlighting marker 605 may have the shape of the transfer device 402. The second highlighting marker 606 may have the shape of the transport system interface 403. The first highlighting marker 605 and the second highlighting marker 606 may have different colors. Markers corresponding to the same unit may have the same color. The highlighting markers 605, 606 may be semitransparent.

FIG. 7 shows a graphical representation of receiving device information 701 step by step by moving the portable device 502 through the laboratory area. FIG. 7 shows a plurality of devices 401. The user 501 may be located closest to the first device 401. The user 501/portable device 502 may thus merely receive the signal 507 broadcasted by the transmitter 506 of the first device 401. The portable device 502 therefore might merely receive information 701 from the first device 401. Signals from other devices may, at this position of the user 501/portable device 502, not be received by the portable device 502. However, when the user 501 carrying the portable device 502 moves from device 401 to device 401, the portable device 502 may also receive information 701 from further devices 401. Thus, by moving the portable device 502 through the laboratory area, device information 701 of several devices 502 may be received step by step by the portable device 502.

FIG. 8 shows a flowchart for an exemplary procedure for selecting two units for pairing. In a first selecting step 801, the user may select a first unit. The selection may be performed via the selection menu 301. The selection may be performed via the selection menu 301 being overlaid with the displayed map of the system 504. In the present example, information indicative of the nearest units, in particular the nearest transfer device 402 and transport system interface 403 may be overlaid with the map of the system 504. Specifically, the transfer device-ID and the interface-ID corresponding to the transfer device 402 and the transport system interface 403 that are located closest to the portable device 502 may be overlaid with the map of the system 504. The positions of these IDs in the map 504 may correspond to the positions of the corresponding units in the laboratory automation system 407. The IDs shown in the map 504 on the display 508 may be comprised by markers. The user may select a first unit, e.g. by tapping on the first marker corresponding to the first unit on the display. In response to the tapping, a tap symbol 805 may be momentarily shown on the display to indicate the position of the tap and that the tap has been captured. If selection was successful, in a second selecting step 802, the (first) marker of the corresponding unit may change. For example, a symbol or a pictogram, in particular a tick 806, may be added to this marker. In a third selecting step 803, the user 501 may select a second unit in a manner analogous to how the first unit was selected in the first selecting step 801. If the second selection was successful, in a fourth selecting step 804, the (second) marker of the corresponding unit may change. For example, a symbol or a pictogram, in particular a tick 806, may be added to this marker. In reaction to a selection of two different units, e.g. the first transfer device 402 and the first transport system interface 403, those two units may be paired.

FIGS. 9a and 9b show graphical representations of marking information. Marking information may comprise a first label 901. In FIG. 9a first units are overlaid with the first label 901 (on the map 504 shown on the display 508 of the portable device 502). The first label 901 may be comprised by a marker. Only successfully paired units may be overlaid with such first labels 901. The first label 901 may comprise information indicative of the units that are paired. The first label 901 may comprise information indicative of whether or not the paring was successful. The first label 901 may comprise IDs of units that are/should be paired. The label 901 may be located at the corresponding units (in the displayed map 504). The corresponding units may be the first units. The first label 901 may merely overlay the first units.

FIG. 9b shows a combined first marker 902. The combined first marker 902 may be indicative of paired first units, in particular that the first transfer device 402 and the first transport system interface 403 are successfully paired. The first marker 902 may have a specific color (e.g., green or blue). The form of the combined first marker 902 may correspond to the corresponding units. The form of the combined first marker 902 may be equal to the corresponding units (that are successfully paired). The position of the combined first marker 902 may correspond to/may be the position of the corresponding units. The combined first marker 902 may comprise a symbol or a pictogram, in particular a tick. In addition or alternative, a pairing list 903 may be provided. The pairing list 903 may be indicative of successfully paired units. The list may comprise information indicative of the paired units. The list may group the paired units, e.g. the first units may be comprised by a first group 904. Two units that are generally linked but not linked to each other cannot be included in the same group. The first group may comprise information indicative of the first units. The first group may comprise information indicative of the first device 401, the first transfer device 402, the first transport system interface 403, the first section of the transport system 404, and/or the first transport module 405. The first group may comprise the name of the first device 401, the device-ID of the first device 401, the transfer device-ID of the first transfer device 402, the interface-ID of the first transport system interface 403, a section-ID of the first section of the transport system 404, and/or the module-ID of the first transport module 405.

FIG. 10 shows an exemplary procedure for pairing a remaining unit 1001. According to the displayed map 504 in the first step of FIG. 10, all units except one are successfully paired. The paired units are marked with first markers. The paired units are marked with a tick. In the exemplary embodiment of FIG. 10, the pairing information may be transmitted to the central backend device and/or the transport management system 406 only if all units are paired. Thus, in the first step of FIG. 10, the pairing information cannot be transmitted to the central backend device and/or the transport management system 406. In the case illustrated in the first step of FIG. 10, the user 501 cannot pair the remaining unit 1001. This is, because the user 501 is at a first location 503.1 too far away from the non-paired unit 1001. Hence, the non-paired unit 1001 is not comprised by the selection menu. The non-paired unit 1001 cannot be selected on the displayed map 504, since the user is too far away from the non-paired unit 1001. According to the second step illustrated in FIG. 10, the user 501 with the portable device 502 moves to a second location 503.2 in the vicinity of the remaining non-paired unit 1001. In reaction, the user 501 may pair the remaining unit 1001 via the portable device 502. This is, because the non-paired unit 1001 may be comprised by the selection menu, since the portable device 502 is in the vicinity of the remaining unit 1001. The non-paired unit 1001 may be selected on the displayed map 504, since the portable device 502 is in the vicinity of the remaining unit 1001. In reaction to the selection the nonpaired unit 1001, the non-paired unit 1001 may be paired. (Only) after the remaining unit 1001 is paired, the paring information may be transmitted to the central backend device and/or the transport management system 406.

1. A method for operating a laboratory automation system, wherein the laboratory automation system comprises
a plurality of devices provided in a laboratory area configured for at least one of sample pre-analytics and sample analysis;
a transport system having a plurality of carriers configured to carry one or more sample containers and configured to transport the plurality of carriers to the plurality of devices;
a plurality of transfer devices each assigned to at least one of the plurality of devices and configured to transfer at least one of a carrier from the plurality of carriers and a sample container received in the at least one carrier between the transport system and the at least one device;
a portable device having one or more data processors and a user interface comprising a display configured to output image data, and an input device configured to receive user input; and
a plurality of data communication modules configured for wireless data communication, wherein each of the plurality of data communication modules is assigned to at least one of a device from the plurality devices, the transport system, a transfer device from the plurality of transfer devices, and the portable device;
the method comprising:
receiving device information for the plurality of devices in the portable device, the device information being indicative of a device identification;
receiving a first user input in the portable device, the user input being indicative of a selection of a first device from the plurality of devices;
in response to receiving the first user input, pairing the first device with the transport system for data communication in operation of the first device for at least one of sample pre-analytics and sample analysis; and
providing first pairing information in the portable device, the first paring information being indicative of the first device and the distribution system being paired successfully.

2. Method of claim 1, wherein the pairing comprises pairing a first transfer device assigned to the first device with the transport system for data communication in operation of the first device for at least one of sample pre-analytics and sample analysis.

3. Method of claim 1, wherein the pairing comprises pairing the first device with a first transport system interface of the transport system, thereby, assigning the first transport system interface to the first device.

4. Method of claim 3, wherein the pairing comprises pairing the first transfer device with the first transport system interface for data communication in operation of the first device for at least one of sample pre-analytics and sample analysis.

5. Method of claim 1, further comprising assigning a first interface identification to the first transport system interface, wherein the first pairing information is indicative of a first device identification assigned to the first device and the first interface identification.

6. Method of claim 5, further comprising assigning the first interface identification to the first transport system interface and a first transfer device identification to the first transfer device, wherein the first pairing information is indicative of the first interface identification and the first transfer device identification.

7. Method of claim 1, wherein the receiving of the device information comprises receiving device information being indicative of a device location in the laboratory area.

8. Method of claim 7, the receiving of the first user input comprises
determining a local position of the portable device within the laboratory area;
providing a device selection menu indicative of one or more devices from the plurality of devices, the one or more devices being located in the vicinity of the local position of the portable device;
outputting the device selection menu via the display; and
in the portable device, receiving a first user input indicative of a selection of the first device from the one or more devices provided in the device selection menu.

9. Method of claim 3, the receiving of the first user input comprises
providing an interface selection menu indicative of one or more transport system interfaces available for pairing with the one or more devices from the device selection menu;
outputting the interface selection menu via the display; and
in the portable device, receiving a first interface user input indicative of a selection of the first transport system interface from the one or more transport system interfaces provided in the interface selection menu.

10. Method of claim 1, wherein receiving of the device information comprises receiving device signals indicative of device information and broadcasted by the plurality of devices while the portable device is moving through the laboratory area.

11. Method of claim 1, further comprising outputting a laboratory layout via the display, the laboratory layout being indicative of the arrangement of the plurality of devices and the transport system in the laboratory area.

12. Method of claim 11, wherein the outputting comprise outputting the laboratory layout with first marking information being indicative of one or more paired devices from the plurality of devices being successfully paired with the transport system.

13. Method of claim 1, further comprising
providing protocol information in the portable device, the protocol information being indicative of the one or more paired devices; and
transmitting the protocol information to a central backend device in response to user confirmation input received in the portable device.

14. A laboratory automation system, comprising
a plurality of devices provided in a laboratory area configured for at least one of sample pre-analytics and sample analysis;
a transport system having a plurality of carriers configured to carry one or more sample containers and configured to transport the plurality of carriers to the plurality of devices;
a plurality of transfer devices each assigned to at least one of the plurality of devices and configured to transfer at least one of a carrier from the plurality of carriers and a sample container received in the at least one carrier between the transport system and the at least one device;
a portable device having one or more data processors and a user interface comprising a display configured to output image data, and an input device configured to receive user input; and
a plurality of data communication modules configured for wireless data communication, wherein each of the plurality of data communication modules is assigned to at least one of a device from the plurality devices, the transport system, a transfer device from the plurality of transfer devices, and the portable device;
wherein the laboratory automation system is configured to:
receive device information for the plurality of devices in the portable device, the device information being indicative of a device identification;
receive a first user input in the portable device, the user input being indicative of a selection of a first device from the plurality of devices;
in response to receiving the first user input, pair the first device with the transport system for data communication in operation of the first device for at least one of sample pre-analytics and sample analysis; and
provide first pairing information in the portable device, the first paring information being indicative of the first device and the distribution system being paired successfully.

15. A laboratory in-vitro diagnostic system, comprising the laboratory automation system of claim 14.
